(19) United States
(12) Patent Application Publication
(10) Pub. No.: US 2019/0194421 A1
(43) Pub. Date: Jun. 27, 2019

(54) 3(2H-)-FURANONE BASED ANTIOXIDANT PACKAGING FILMS

(71) Applicant: Bemis Company, Inc., Neenah, WI (US)

(72) Inventor: Ankush A. Gokhale, Menasha, WI (US)

(21) Appl. No.: 16/325,485

(22) PCT Filed: Aug. 15, 2016
(86) PCT No.: PCT/US16/47010

§ 371 Date: Feb. 14, 2019

(51) Int. Cl. C08K 5/1535; C07D 307/32; B32B 27/18; B32B 27/32; B32B 27/30; B65D 65/40; B65D 65/42

(52) U.S. Cl. CPC C08K 5/1535; C07D 307/32; B32B 27/18; B32B 27/32; B32B 27/306; B32B 2270/00; B65D 65/42; B32B 2553/00; B32B 2307/714; B32B 2250/242; B32B 2250/246; B65D 65/40

(57) ABSTRACT

The present invention is directed to packaging films comprising an antioxidant based on 3(2H-)-furanone having a general structural formula (I): R₁═CH₃, CH₂CH₃; R₂═H, CH₃; R₃═H, CH₂CH₃.

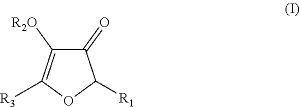

### BACKGROUND OF THE INVENTION

The present invention relates generally to primary packaging, and in particular to packaging films containing an antioxidant that significantly extends the shelf-life of products packaged therein.

The following description of the background and embodiments of the invention thereafter is provided to aid in understanding the invention, but is not admitted to describe or constitute prior art to the invention. The contents of the articles, patents, and patent applications, and all other documents and electronically available information mentioned or cited in this application, are hereby incorporated by reference in their entirety to the same extent as if each individual publication was specifically and individually indicated to be incorporated by reference, including any references cited in the articles, patents, patent applications and documents cited herein, except to the extent they may directly contradict the present disclosure. Applicant reserves the right to physically incorporate into this application any and all materials and information from any such articles, patents, patent applications, or other documents

Oxidation is one of the most serious problems the food industry faces in protecting shelf stable foods due to its deteriorating effects on food quality. The major food quality issues include decreased nutritional quality, increased toxicity, development of off-odor, and altered texture and color. The initial products of autoxidation are hydroperoxides, which are colorless, tasteless, and odorless. However, these hydroperoxides decompose to low-molecular-weight compounds that result in the development of rancid flavors and odors. Several food additive ingredients and food packaging strategies are currently employed to prevent these harmful oxidative reactions within food systems.

Oxidation remains a problem to the food industry because of some rather well-known challenges including the difficulty in removing environmental oxygen completely from foods and the package. It remains very difficult to remove the entire headspace oxygen and that dissolved or trapped within the beverage or food composition. While vacuum packaging is effective, it can be difficult to use with many foods, especially cereal products.

BHT-impregnated films are being used in cereal packaging with the intended purpose of migration of this compound out of the cereal liner and into the product. Butylated hydroxytoluene (BHT) is an FDA-approved food ingredient that is commonly used as a primary antioxidant. This antioxidant significantly extends the shelf life of foods containing lipids susceptible to oxidation such as vegetable oils, animal fats, flavorings, spices, nuts, processed meats and snack products. However, while there is no scientific evidence that BHT is harmful in the amounts used in cereal liners, nevertheless, there is a perception that this compound may be harmful to humans. Consequently, major cereal producers have begun removing BHT from their cereal packaging. Therefore, a need exists in the packaging industry to develop new packaging materials which inhibit the formation of harmful oxidation products, and extend the shelf life of foods.

### SUMMARY

At least some embodiments of the present invention are directed to packaging films that inhibit the onset of oxidative spoilage of products packaged in these films. Towards this end, packaging films are described herein having an antioxidant. Release of antioxidant from the film is by volatilization, direct contact with the packaged product, or by migration from a core layer to a product-contact layer and volatilization and/or direct contact with the packaged product.

Provided are packaging films comprising a volatile antioxidant present in or on a layer of the film which extends the product's shelf-life. As used herein, the term “volatile” refers to a substance having a vapor pressure of greater than 1.0E-05 mmHg (1.32E-08 atm) at 25° C. Vapor pressure or equilibrium vapor pressure is the pressure of a vapor in thermodynamic equilibrium with its condensed phases in a closed container. All liquids and solids have a tendency to evaporate into a gaseous form, and all gases have a tendency to condense back to their liquid or solid form. Determination of vapor pressure of liquids and solids are known in the art and can be measured without undue experimentation. The Knudsen effusion method vis-à-vis a dynamic gravimetric technique is an example of one method of determining vapor pressure of an antioxidant. This technique can be performed automatically by a vapor pressure analyzer supplied by Surface Measurement Systems, Ltd., London, UK.

Provided herein are packaging films comprising an antioxidant having a designation of generally recognized as safe (GRAS) by the United States Food and Drug Administration (FDA).

The packaging films may be a monolayer film comprising an antioxidant, a multilayer film comprising an antioxidant, or either a monolayer or multilayer film each with a coating of a layer comprising an antioxidant. The antioxidant may be present in any film layer of the packaging film. The antioxidant may be present in more than one layer, including in all layers, of the packaging film.

The packaging films may include any number of layers as needed depending upon the requirements of a particular packaging application. These additional layers may include, but are not limited to oxygen barrier layers, moisture barrier layers, chemical barrier layers, abuse layers, tie or adhesive layers, bulk layers, and odor and oxygen scavenging layers. It is contemplated that the layer comprising an antioxidant can be combined with many different materials such as, but not limited to, plastics, papers, non-woven materials, metal foils to form various packaging structures. In some embodiments, the packaging films are considered oxygen barrier films and have an oxygen transmission rate (O₂TR) value of less than or equal to 10 cm³/100 in²/24 hours at 1 atmosphere, 23° C. and 0% RH.

The packaging films can be converted into various packaging configurations, including but not limited to cereal liners, bags, pillow pouches, stand-up pouches, quad pouches, zipped pouches, over-wraps, lidding films, thermoformed trays, vacuum packages, vacuum skin packaging and the like.

As used herein, the term “product-contact layer” refers to an interior layer of a package which is in direct contact with a packaged product. Typically, the product-contact layer may also include a coating on the inner surface of a packaging film. The thickness of the product-contact layer can be selected as desired, but may typically be in a range from 2.54 μm to 1270 μm (0.1 mil to 50 mil), or from 12.7 μm to 254 μm (0.5 mil to 10 mil), or from 25.4 μm to 127 μm (1 mil to 5 mil). In contrast, the term “core layer” as used herein refers to any film layer that is not in direct contact with a product. In some embodiments, the core layer is sandwiched between two layers of the film structure. In other embodiments, a core layer is an exterior layer opposite the product-contact layer.

Also provided herein are packages, especially for cereal foodstuffs. In some embodiments, food packages in the form of cereal liners are provided.

Further provided herein are methods for extending the shelf-life and/or the sell-by date of a food product. The methods comprise: obtaining any packaging film having a layer comprising an antioxidant as disclosed herein: and packaging a food product within the packaging film.

### DETAILED DESCRIPTION

The Antioxidant

The antioxidant is based on 3(2H-)-furanone having a general structural formula:

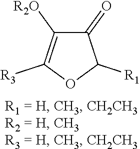

In embodiments where R₁ and R₂ are each hydrogen and R₃ is a methyl group, the antioxidant is 4-hydroxy-5-methyl-3(2H)-furanone.

In embodiments where R₁ and R₃ are each a methyl group and R₂ is a hydrogen, the antioxidant is 4-hydroxy-2,5-dimethyl-3(2H)-furanone. This antioxidant is commonly known as furaneol and has a vapor pressure of 3.2E-02 mmHg (4.2E-05 atm) at 25° C. Furaneol also has a designation of generally recognized as safe (GRAS) by the United States Food and Drug Administration (FDA).

In embodiments where R₁ is an ethyl group, R₂ is hydrogen and R₃ is a methyl group, the antioxidant is 2-ethyl-4-hydroxy-5-methyl-3(2H)-furanone.

In embodiments where R₁ is a methyl group, R₂ is hydrogen and R₃ is an ethyl group, the antioxidant is 5-ethyl-4-hydroxy-2-methyl-3(2H)-furanone. This antioxidant is commonly known as ethyl furaneol and has a vapor pressure of 1.2E-02 mmHg (1.6E-05 atm) at 25° C.

In embodiments where R₁ and R₃ are each a methyl group and R₂ is a methyl group, the antioxidant is 4-methoxy-2,5-dimethyl-3(2H)-furanone.

In embodiments where R₁ and R₃ are each an ethyl group and R₂ is hydrogen, the antioxidant is 4-hydroxy-2.5-diethyl-3(2H)-furanone.

In some embodiments, the antioxidant can be a mixture of two or more antioxidants of different composition, such as any combination or blend of the specific antioxidants described above. Alternatively, the antioxidant may be homogeneous in nature, e.g., composed of substantially only one specific antioxidant composition. In some embodiments, the antioxidant is incorporated into a film layer. The antioxidant may be incorporated into any layer of the film. In some embodiments, the amount of antioxidant incorporated into this layer may be within the range from 5.0E-04 g/m² to 10 g/m², or from 5.0E-03 g/m² to 5 g/m², or from 5.0E-02 g/m² to 2.5 g/m² relative to the total weight of this layer. There are several methods which can be used to incorporate the antioxidant into a film layer. All the components of the layer may be dry blended in the required weight ratio in a suitable device such as a tumble blender. The resulting dry blend may then be melted in suitable equipment such as an extruder. Alternatively, a masterbatch can be prepared by metering the layer components directly into a single- or twin-screw extruder. The specific conditions for operating a single-screw extruder will differ from that of a twin-screw extruder, but those skilled in the art can readily determine the necessary operating conditions needed to prepare masterbatches suitable for use with the present invention.

In other embodiments, the antioxidant is coated onto a film layer. A coating can be achieved by first blending the antioxidant with a polymer composition, then extrusion coating the blend onto a substrate. In some embodiments, the amount of antioxidant on this layer may be within the range from 5.0E-04 g/m² to 10 g/m², or from 5.0E-03 g/m² to 5 g/m², or from 5.0E-02 g/m² to 2.5 g/m² relative to the total weight of this layer. In such embodiments, other ingredients may be added to the polymer composition to facilitate the dispersion of antioxidant in the polymer composition. The substrate can be a monolayer or a multilayer film. In some of these embodiments, the blended antioxidant/polymer composition forms the product-contact layer of the packaging film. Extrusion coating is well-known in the art and can be performed without undue experimentation. In some embodiments, the antioxidant may be dissolved or emulsified in a material and then the solution or emulsion applied onto the substrate surface. The substrate surface is a film layer. In some embodiments, the coated substrate may be a product-contact and/or core layer of a multilayer film structure. In such embodiments, the multilayer structure can be a laminated film formed by extrusion lamination. In some embodiments, the antioxidant solution or emulsion may be dried, cured and/or partially or fully removed by evaporation. Such methods may involve printing technology which is also known to those skilled in the art. In such embodiments, the substrate can also be a monolayer or a multilayer film. Alternatively, the antioxidant may be applied neat to a film layer surface without a solvent or other carrier material. In any of the aforementioned methods, coating such as by gravure coating, roll coating, dipping and/or spraying may be used.

The Effectiveness of Antioxidant

The free radical scavenging capability of the antioxidant was determined by its reaction with 1,1-diphenyl-2-picrylhydrazyl (DPPH). DPPH is a common assay technique that is frequently used in measuring antioxidant activity. In this reaction, the DPPH undergoes reduction in the presence of an antioxidant compound and this reduction can be detected by a change in color in the visible spectrum. Spectroscopic analysis was used to measure the change in color, and the measured color change was directly proportional to the antioxidant capability. The antioxidant was added directly to a solution of DPPH in methanol/ethanol/isopropanol to a concentration of 12.3 ppm and allowed to react for 45 minutes at 25° C. in the dark. The percentage of DPPH free radical scavenged is listed below in TABLE 1.

TABLE 1

| | % of DPPH Free Radicals |
|---|---|
| Antioxidant | Scavenged |
| Control (no antioxidant) | 0.5 |
| BHT | 7.5 ± 0.6 |
| Maltol | 16.6 ± 0.2 |
| Furaneol | >95 |

The effectiveness of the antioxidant was also determined by incorporating between 2.5 and 3 weight % of an antioxidant into a product-contact (sealant) layer comprising ethylene-based hexene plastomer. A 60 cm² sample of the film was cut and suspended in a closed glass container above a solution containing 1,1-diphenyl-2-picrylhydrazyl (DPPH) in isopropanol. There was no contact between the DPPH solution and film. Scavenging of the free radicals present in the DPPH solution occurred by volatilization of antioxidant from the film and its subsequent dissolution into the DPPH solution. Spectroscopic analysis was used to measure the change in the color, which was directly proportional to the antioxidant capability. The results of this measurement are reported in TABLE 2 below:

TABLE 2

| | % of DPPH Free Radicals Scavenged | | |
|---|---|---|---|
| # of Days | BHT | Maltol | Furaneol |
| 1 | 75 ± 5 | 35 ± 2.1 | 65 ± 3.2 |
| 3 | >95 | 95 ± 3 | 92 ± 2.5 |
| 10 | >95 | >95 | >95 |

The Polymer Composition

The polymer composition may comprise any plastic material. In some embodiments, the polymer composition may include a non-polar macromolecule. The polymer composition may be used to form a product-contact layer or a core layer of a packaging film. In such embodiments, the polymer composition may comprise polyethylene including but not limited to ultra-low density polyethylene, low density polyethylene, linear low density polyethylene, metallocene polyethylene, medium density polyethylene, high density polyethylene, polypropylene, polybutylene, and copolymers of either polyethylene or polypropylene. In some embodiments, the polymer composition may comprise a mixture of non-polar macromolecules. If the polymer composition is used in a product-contact layer, the non-polar macromolecule or blends thereof may be heat sealable.

Multilayer packaging films having an antioxidant in at least one layer as described herein may be fabricated by blown film coextrusion methods. Other conventional coextrusion methods can also be used including slot cast coextrusion, extrusion lamination, extrusion coating and combinations of blown film coextrusion with one or more alternative methods. In a blown film coextrusion embodiment, the multilayer packaging film can be produced using multiple extruders which feed into a multi-manifold circular die head through which the film layers can be forced and formed into a cylindrical multilayer film bubble. The bubble can be quenched, then collapsed and formed into a multilayer film. Blown film extrusion processes are known in the art and have been described, for example, in The Encyclopedia of Chemical Technology, Kirk-Othmer, 3rd ed., John Wiley & Sons, New York, 1981, Vol. 16, pp. 416-417 and Vol. 18, pp. 191-192.

Provided below are non-limiting prophetic examples of three different multilayer packaging films, each of which can have at least one layer that can incorporate at least one of the disclosed antioxidants. The layer compositions are described in TABLES 3-5 below. In each of the three prophetic film examples, the antioxidant is identified in bold text. In these examples, the interior product-contact (sealant) layer and/or other layers may control the rate of migration of antioxidant from a core layer to the interior surface of the film. Each of these prophetic film examples can be readily configured to form a bag or other package for storing or holding cereal foodstuffs or other suitable foods, as disclosed herein.

TABLE 3
Multilayer Packaging Film Example 1
Total thickness = 57.15 μm (2.25 mil)

| | |
|---|---|
| Layer 1 | 88.0 wt. % of Ionomer + 12.0 wt. % Additives |
| (Product- | [Basis wt. = 4.11] |
| contact/ | |
| Sealant) | |
| Layer 2 | 95.0 wt. % of high density polyethylene (HDPE) + 4.5 wt. % |
| | of a polyethylene + 0.5 wt. % of antioxidant |
| | [Basis wt. = 23.03] |
| Layer 3 | 96.0 wt. % of high density polyethylene (HDPE) + 3.2 wt. % |
| | of linear low density polyethylene (LLDPE) + 0.8 wt. % of |
| | additives |
| | [Basis wt. = 6.88] |

TABLE 4
Multilayer Packaging Film Example 2

| | |
|---|---|
| Layer 1 | 54.2 wt. % of ethylene vinyl acetate copolymer (EVA) + |
| (Product- | 30 wt. % of ultra-low density polyethylene (ULDPE) + |
| contact/ | 12 wt. % polybutylene (PB) + 3.8 wt. % additives |
| Sealant) | [Basis wt. = 5.13] |
| Layer 2 | 64.0 wt. % of linear low density polyethylene (LLDPE) + |
| | 25 wt. % anhydride modified polyethylene + 6.6 wt. % of |
| | antioxidant + 4.4 wt. % of linear low density polyethylene |
| | (LLDPE) |
| | [Basis wt. = 2.2] |
| Layer 3 | 80 wt. % of ethylene vinyl alcohol copolymer (EVOH) + |
| | 20 wt. % of linear low density polyethylene (LLDPE) |
| | [Basis wt. = 2.71] |
| Layer 4 | 83.75 wt. % of linear low density polyethylene (LLDPE) + |
| | 16.25 wt. % anhydride modified polyethylene |
| | [Basis wt. = 1.89] |
| Layer 5 | 92 wt. % of high density polyethylene (HDPE) + 8 wt. % of |
| | low density polyethylene (LDPE) |
| | [Basis wt. = 21.07] |

TABLE 5
Multilayer Packaging Film Example 3
Total thickness = 44.45 μm (1.75 mil)

| | |
|---|---|
| Layer 1 | 93.0 wt. % of Ionomer + 7.0 wt. % Additive |
| (Product- | [Basis wt. = 3.20] |
| contact/ | |
| Sealant) | |
| Layer 2 | 82.0 wt. % of high density polyethylene (HDPE) + 15.3 wt. |
| | % polyethylene + 1.7 wt. % antioxidant + 1 wt. % additives |
| | [Basis wt. = 3.69] |
| Layer 3 | 85.0 wt. % of linear low density polyethylene (LLDPE) + |
| | 15.0 wt. % anhydride modified polyethylene |
| | [Basis wt. = 2.34] |
| Layer 4 | 100 wt. % of nylon |
| | [Basis wt. = 2.45] |
| Layer 5 | 85.0 wt. % of linear low density polyethylene (LLDPE) + |
| | 15.0 wt. % anhydride modified polyethylene |
| | [Basis wt. = 2.09] |
| Layer 6 | 75 wt. % of high density polyethylene (HDPE) + 25 wt. % |
| | nylon |
| | [Basis wt. = 5.5] |
| Layer 7 | 95 wt. % of high density polyethylene (HDPE) + 1.2 wt. % of |
| | linear low density polyethylene (LLDPE) + 3.8 wt. % |
| | additives |
| | [Basis wt. = 7.25] |

The above description and examples illustrate certain embodiments of the present invention and are not to be interpreted as limiting. Selection of particular embodiments, combinations thereof, modifications, and adaptations of the various embodiments, conditions and parameters normally encountered in the art will be apparent to those skilled in the art and are deemed to be within the spirit and scope of the present invention.

1. A multilayer packaging film comprising:
a layer comprising:
i) a polymer composition; and
ii) an antioxidant based on 3(2H-)furanone having a general structural formula:

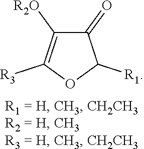

2. The multilayer packaging film according to claim 1, wherein the antioxidant comprises 4-hydroxy-5-methyl-3(2H)-furanone.

3. The multilayer packaging film according to claim 1, wherein the antioxidant comprises 4-hydroxy-2,5-dimethyl-3(2H)-furanone.

4. The multilayer packaging film according to claim 1, wherein the antioxidant comprises 2-ethyl-4-hydroxy-5-methyl-3(2H)-furanone.

5. The multilayer packaging film according to claim 1, wherein the antioxidant comprises 5-ethyl-4-hydroxy-2-methyl-3(2H)-furanone.

6. The multilayer packaging film according to claim 1, wherein the antioxidant comprises 4-methoxy-2,5-dimethyl-3(2H)-furanone.

7. (canceled)

8. The multilayer packaging film according to claim 1, wherein the antioxidant has a vapor pressure of greater than 1.0E-05 mmHg (1.32E-98 atm) at 25° C.

9. The multilayer packaging film according to claim 1, wherein the antioxidant has a vapor pressure of greater than 1.0E-04 mmHg (1.32E-07 atm) at 25° C.

10. The multilayer packaging film according to claim 1, wherein the antioxidant has a vapor pressure of greater than 1.0E-03 mmHg (1.32E-06 atm) at 25° C.

11. The multilayer packaging film according to claim 1, wherein the polymer composition comprises a non-polar macromolecule.

12. The multilayer packaging film according to claim 11, wherein the non-polar macromolecule comprises polyethylene.

13. The multilayer packaging film according to claim 11, wherein the non-polar macromolecule comprises a mixture comprising high density polyethylene.

14. The multilayer packaging film according to claim 1, wherein the layer is a core layer of the multilayer packaging film.

15. The multilayer packaging film according to claim 1, wherein the layer is a product-contact layer of the multilayer packaging film.

16. The multilayer packaging film according to claim 1, wherein the antioxidant is incorporated into the polymer composition.

17. The multilayer packaging film according to claim 1, wherein the antioxidant is applied to a surface of the polymer composition.

18. (canceled)

19. (canceled)

20. The multilayer packaging film according to claim 15, wherein the product-contact layer is a coating.

21. The multilayer packaging film according to claim 1, wherein the antioxidant is present in an amount within a range from 5.0E-04 g/m² to 10 g/m² relative to a total weight of the layer.

22. The multilayer packaging film according to claim 1, wherein the antioxidant is present in an amount within a range from 5.0E-02 g/m² to 5 g/m² relative to the total weight of the layer.

23. The multilayer packaging film according to claim 1, wherein the multilayer packaging film is a cereal liner.

24. (canceled)
